(19) United States
(12) Patent Application Publication
(10) Pub. No.: US 2022/0154247 A1
(43) Pub. Date: May 19, 2022

(54) ULTRASENSITIVE METHOD FOR DETECTING CELL DEATH

(71) Applicants: INSERM (Institut National de la Santé et de la Recherche Médicale), Paris (FR); Université Paul Sabatier Toulouse III, Toulouse (FR)

(72) Inventors: Henrik LAURELL, Ramonville (FR); Eric LACAZETTE, Goyrans (FR); Christian TOURIOL, Pechbusque (FR); Jason IACOVONI, Leguevin (FR)

(21) Appl. No.: 17/439,599

(22) PCT Filed: Mar. 16, 2020
(86) PCT No.: PCT/FR2020/050571

§ 371 Date: Sep. 15, 2021

(30) Foreign Application Priority Data
Mar. 18, 2019 (FR) 19 02755

(51) Int. Cl. C12Q 1/6806; C12Q 1/686; G01N 33/50

(52) U.S. Cl. CPC C12Q 1/6806; G01N 33/5014; C12Q 1/686

(57) ABSTRACT

The invention relates to a method for detecting cell death using PCR (polymerase chain reaction) techniques, including qPCR/quantitative PCR or ddPCR/digital droplet PCR, or any other technique for detecting a small amount of DNA (such as nanostrings).

### TECHNICAL FIELD

The invention relates to a method for detecting cell death by the use of PCR techniques (polymerase chain reaction): qPCR, i.e. quantitative PCR; ddPCR, i.e. droplet digital PCR, etc.) or any other technique for detecting small amounts of DNA (Nanostring, etc.).

### PRIOR ART

Cell death is the irreversible cessation of vital functions, with modification of structures on a cellular and molecular level. This process of cell death can occur in a number of relatively different ways (Gallouzzi et al., Cell Death Differ, 2018).

A distinction will mainly be made between cell death via apoptosis, or regulated cell death, which takes place according to a sequence of finely regulated events and is initiated in response to a death signal (oxidative stress, exogenous stress, DNA damage, viral infection, and the like) or programmed cell death involved in the maintenance of tissue homeostasis where each cell possesses a more or less determined lifetime.

Death by necrosis for its part results from cellular processes generally triggered “accidentally”, such as for example freezing, heat, mechanical lesion, etc. The choice of the response by apoptosis or necrosis can also depend on the intensity of the attack.

In mechanistic terms, during death by apoptosis the following are observed: alterations of the membrane, enzyme signals (such as the activation of caspase proteins), condensation of the nucleus and of the cytoplasm, aggregation of chromatin, mitochondrial damage and the fragmentation of nuclear DNA into internucleosomal fragments. Interestingly, most antitumor agents can induce an apoptosis phenomenon.

Conversely, the morphological criteria of necrotic cells are different. Destructuring of the cell membrane will cause a massive influx of water into the cell, destruction of the intracellular organelles, which leads to the release of lytic enzymes from the lysosomes and the peroxisomes, resulting in the digestion of the cell, degradation of the DNA and then cell death.

Although the pathways leading to cell death by apoptosis or by necrosis are very different in terms of mechanisms, the following has been observed in each of these pathways: degradation of the DNA, preceded by single-strand and double-strand breaks, and generation of high molecular weight DNA fragments which are progressively degraded into internucleosomal fragments (apoptosis) or progressively degraded randomly (necrosis).

The methods for detecting apoptosis can be categorized into 4 main principles depending on whether they detect i) alteration of the plasma membrane, ii) activation of caspases, iii) mitochondrial damage, or iv) fragmentation of the DNA.

Regarding the activation of the caspases, it is possible to measure the caspase activity by the fusion of a tetrapeptide site of cleavage by the caspases to a fluorometric or luminescent reporter (mention will be made of the Caspase-Glo® kit from Promega). This method has the advantage of being fast, simple to use and provided by numerous suppliers. However, this method is expensive, requires a spectrofluorometer or luminometer and only allows detection of caspase-dependent apoptosis.

For the detection of alteration of the plasma membrane, detection of the phosphatidylserines exposed in the outer layer of the plasma membrane is also proposed by a large number of suppliers (mention will be made of Pacific Blue™ Annexin V from BioLegend®). The principle is the use of annexin V (a protein which binds specifically to the phosphatidylserines in a calcium-dependent manner), coupled to different (mainly fluorescent) reporters enabling the detection of said protein. The analysis is carried out by flow cytometry (FACS) or by microscopy. This widely proposed method offers a wide choice of fluorochromes and makes it possible to detect apoptotic and necrotic cells. However, this method requires a flow cytometer and the quantification of the apoptosis of adherent cells in flow cytometry is very delicate. The use of microscopy can overcome this problem. However, quantification is more laborious. This method is therefore of little suitability for adherent cells.

Among the methods which detect the fragmentation of DNA, mention may first of all be made of a method for visualizing fragmented DNA after gel electrophoresis followed by visualization by an intercalating agent. The principle consists in extraction of the genomic DNA and then separation of the fragments by agarose gel electrophoresis (Apoptotic DNA Ladder Kit). Although this method has the advantage of having a low cost and of not requiring specific and expensive apparatuses, this method is extremely insensitive.

Mention will also be made of a method for quantifying cytoplasmic DNA coupled to histones by ELISA and the use of antibodies against DNA and histones (Cell Death Detection ELISA kit from the company Roche). This method is described for example in the U.S. Pat. No. 5,637,465. While it is quite simple to implement, this method is relatively laborious. Its sensitivity and also the detection range are limited in spite of its high cost.

Hooker et al., 2012 (Nucl Acids Res, 40(15)e113); Hooker et al., 2009 (J Cell Mol Med, 13(5):948-958), and Staley et al., 1997 (Cell Death Differ, 4:66-75) describe a method for detecting fragmented DNA using a “quantitative ligation-mediated PCR” which makes it possible to amplify the fragments with blunt ends that are formed following the activation of nucleases. This laborious method requires multiple steps.

Lastly, Botezatu et al., 2000 (Clin Chem 46(8):1078-1084); Umetani et al., 2006 (Clin Chem 52(6):1062-1069); Lou et al., Int J Mol Med 35: 72-80); Fawzy et al., 2016 (J Egypt Natl Canc Inst, 28: 235-242) describe methods for detecting/quantifying circulating (or free) genomic DNA and more particularly detecting genomic DNA resulting from cell death. These methods involve the amplification of repeat “Alu” sequences by quantitative PCR. In this method, the DNA samples are serum and plasma samples.

There therefore remains a need for developing a method for detecting cell death which is sensitive, simple and rapid, has moderate cost, and works on adherent cells or cells in suspension and even for tissues.

### DISCLOSURE OF THE INVENTION

The present invention relates to a method for detecting cell death based on the detection of genomic DNA fragments of nuclear origin which are present in the cell cytoplasm. Specifically, the inventors have developed a very sensitive method enabling the detection of genomic DNA fragments of nuclear origin in the cytoplasm.

Unlike prior art methods for detecting DNA fragments by PCR, according to which the DNA fragments are detected and quantified in a total cell extract (nucleus+cytoplasm) or else in biological fluids such as plasma, detection is performed on a cytoplasmic extract. This cytoplasmic extract is obtained by incubating the cell sample with a lysis buffer or a hypotonic buffer in order to lyse or permeabilize the plasma membrane without permeabilizing the nuclear membrane.

Detection from cytoplasmic extracts makes it possible to obtain a method which is more rapid, simpler to implement and less expensive compared to the prior art methods.

The cytoplasmic extracts correspond to all or some of the cellular cytoplasm content. When the process of cell death is triggered, the cytoplasmic extract is enriched in genomic DNA of nuclear origin compared to cells which have not initiated the death process.

Detection of the genomic DNA fragments of nuclear origin that are present in the cytoplasm is measured by amplification/detection of a zone present in at least one copy in the genome. Thus, these genomic DNA fragments can advantageously be detected and quantified in a sample. The quantity of DNA fragments recovered is proportional to the quantity of cells that have initiated the death process in the sample, thus making it possible to detect and quantify this process.

The detection of genomic DNA fragments in the cytoplasm, using specific primers targeting repeat sequences present in the genome, confers increased sensitivity on the method according to the invention. The comparative results obtained confirm that the sensitivity is greater than that obtained with existing conventional methods already on the market.

### BRIEF DESCRIPTION OF THE DRAWINGS

Other characteristics, details and advantages of the invention will become apparent on reading the detailed description that follows, and on analyzing the appended figures in which:

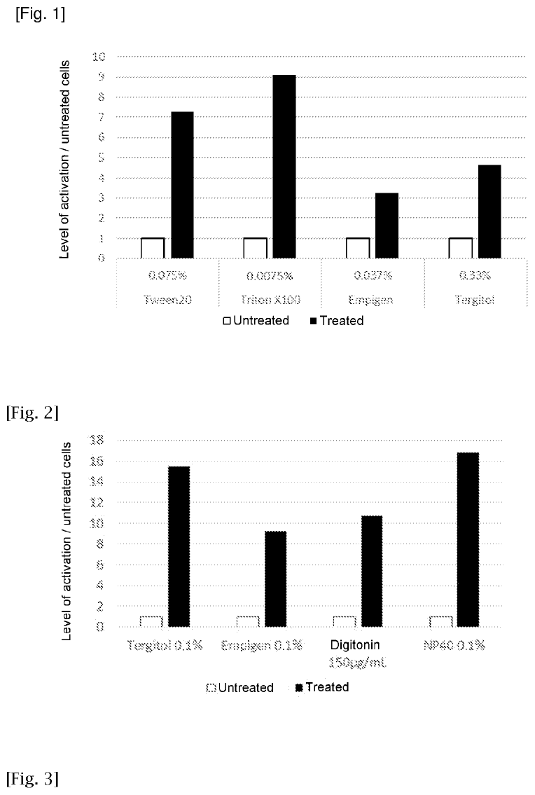

### FIG. 1

FIG. 1 represents the level of activation of cell death realized on cytoplasmic extracts of HepG2 cells obtained using various detergents in the method according to the invention.

### FIG. 2

FIG. 2 represents the level of activation of cell death realized on cytoplasmic extracts of OCI-AML3 cells obtained using various detergents in the method according to the invention.

### FIG. 3

FIG. 3 represents the level of activation of cell death realized on cytoplasmic extracts of OCI-AML3 cells obtained using various detergents in the method according to the invention.

### FIG. 4

FIG. 4 represents the level of activation of cell death realized on cytoplasmic extracts of MDA-MB-231 cells obtained using various detergents in the method according to the invention.

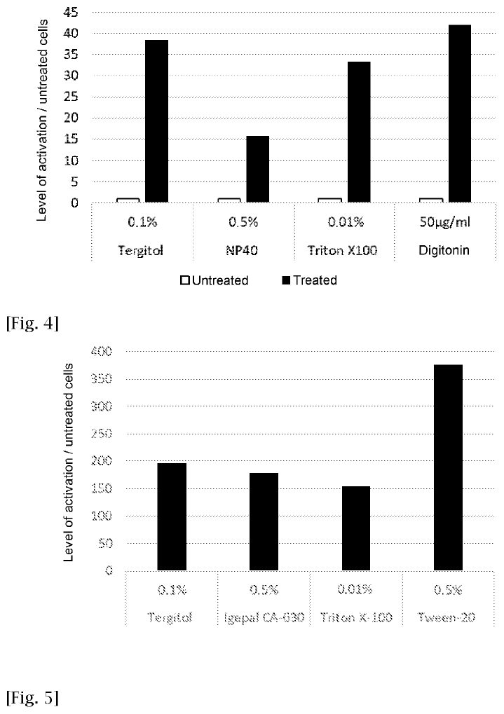

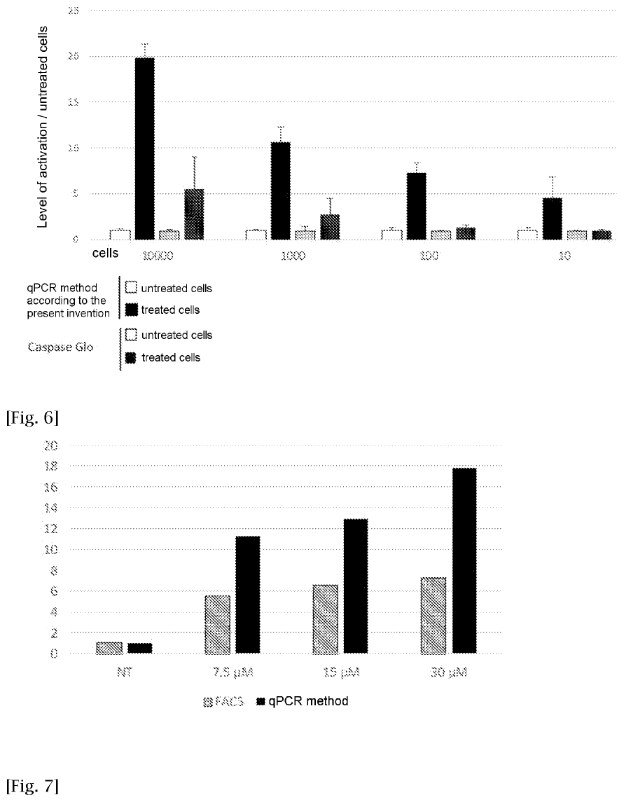

### FIG. 5

FIG. 5 represents the comparison of the sensitivity of the method for detecting cell death by qPCR according to the present invention relative to a prior art Caspase Glo 3/7 method.

### FIG. 6

FIG. 6 represents the comparison of the sensitivity of the method for detecting cell death by qPCR according to the present invention relative to a prior art flow cytometry method (annexin V, propidium iodide labeling).

### FIG. 7

FIG. 7 represents the comparison of the sensitivity of the method for detecting cell death by qPCR (direct lysis) according to the present invention relative to flow cytometry (annexin V, propidium iodide labeling).

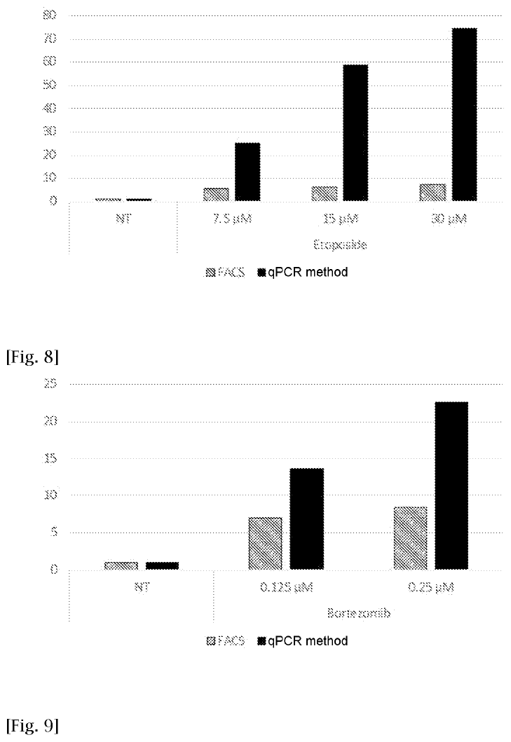

### FIG. 8

FIG. 8 represents the comparison of the sensitivity of the method for detecting cell death by qPCR according to the present invention relative to flow cytometry (annexin V, propidium iodide labeling).

### FIG. 9

FIG. 9 represents the comparison of the sensitivity of the method for detecting cell death by qPCR (direct lysis) according to the present invention relative to flow cytometry (annexin V, propidium iodide labeling).

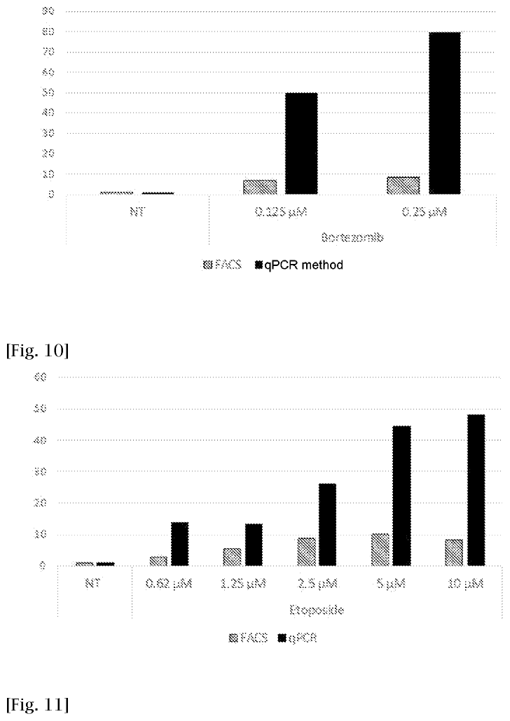

### FIG. 10

FIG. 10 represents the comparison of the sensitivity and also the saturation threshold of the method for detecting cell death by qPCR according to the present invention relative to flow cytometry (annexin V, propidium iodide labeling).

### FIG. 11

FIG. 11 represents the comparison of the sensitivity of the method for detecting cell death by qPCR according to the present invention relative to flow cytometry (annexin V, propidium iodide labeling).

### FIG. 12

FIG. 12 represents the comparison of the sensitivity of the method for detecting cell death by ddPCR according to the present invention on isolated cells.

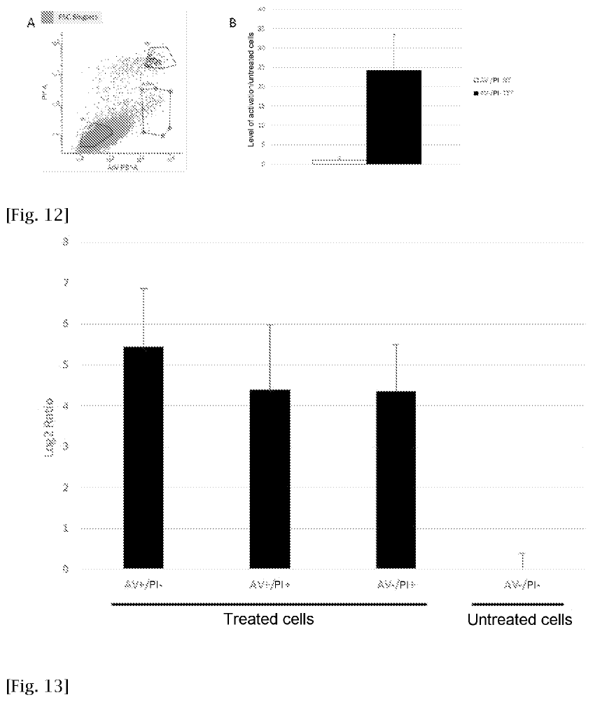

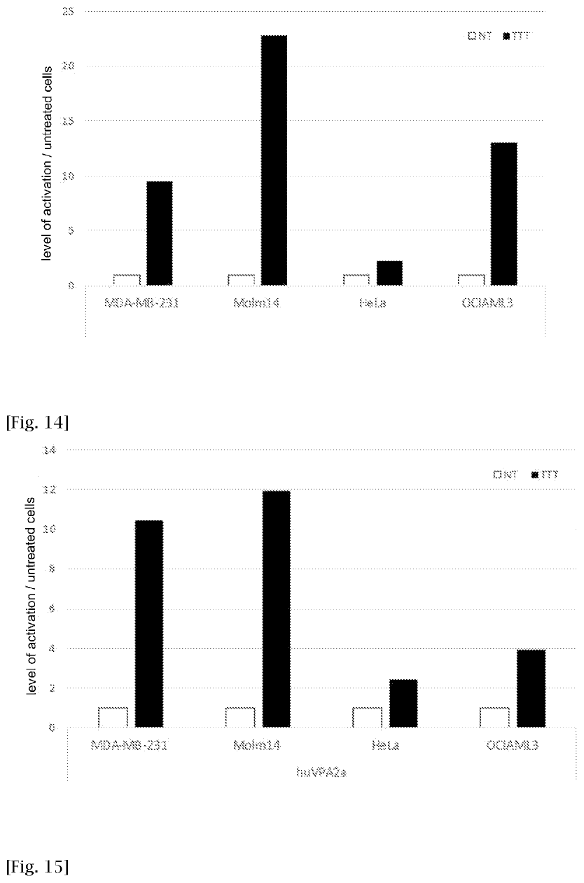

### FIG. 13

FIG. 13 represents the level of activation of cell death realized on cytoplasmic extracts of different cell lines with the aid of a pair of primers targeting a sequence present in one copy per genome.

### FIG. 14

FIG. 14 represents the level of activation of cell death realized on cytoplasmic extracts of different cell lines with the aid of a pair of primers targeting a sequence present in two copies per genome.

### FIG. 15

FIG. 15 represents the level of activation of cell death realized on cytoplasmic extracts originating from the OCI-AML3 cell line with the aid of two pairs of primers each targeting a sequence present in one copy per genome.

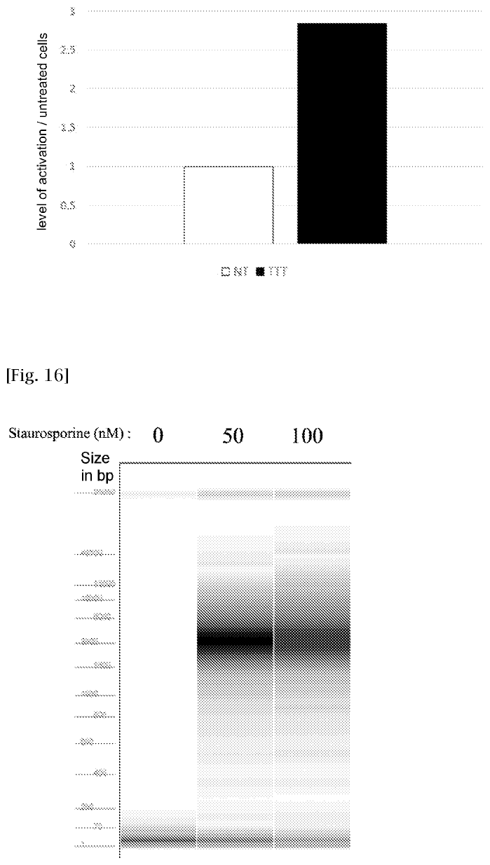

### FIG. 16

FIG. 16 shows an increase in the quantity of DNA fragments in the cytoplasmic fraction which are obtained after extraction with a lysis buffer described in the method according to the invention and are detected by capillary electrophoresis, this being exclusively in cells treated by a drug inducing the death thereof (staurosporine) (MDA 50 and MDA 100) compared to cells not treated with staurosporine (MDA NT).

### DETAILED DESCRIPTION

The drawings and the description hereinafter contain, in the main, elements of a certain nature. They can therefore serve not only for better understanding of the present invention but also contribute to the definition thereof, where appropriate.

The present invention thus relates to a method for quantifying cell death in a cell sample, characterized in that at least one sequence present on genomic DNA fragments of nuclear origin is amplified from the cytoplasmic extract of said sample.

Specifically, the inventors have advantageously exploited the abnormal presence of genomic DNA fragments of nuclear origin that are located in the cytoplasm of the cells during the process of cell death. Fragmented genomic DNA of nuclear origin may thus be detected from cytoplasmic extracts of the cells.

The term “cell death” for the purposes of the present invention is understood to mean cell death by apoptosis and/or cell death by necrosis.

The term “genomic DNA fragments” or “fragmented genomic DNA” is understood to mean the fragments of DNA of nuclear origin that are generated during the process of cell death.

The cell sample may be a sample of cells in in vitro culture, such as adherent cells or cells in suspension, a sample comprising circulating tumor cells, a sample comprising purified circulating tumor cells, a blood sample containing circulating cells or any other sample, such as a plasma sample, a urine sample or a saliva sample.

According to one embodiment, the method for quantifying cell death in a cell sample comprises:
obtaining a cytoplasmic extract from a cell sample;
amplifying at least one sequence in said cytoplasmic extract;
quantifying the genomic DNA detected in said cytoplasmic extract.

The term “cytoplasmic extract” is understood to mean the soluble part of the cell cytoplasm, also referred to as cytosol, which is recovered after the specific permeabilization of the plasma membrane without alteration of the nuclear membrane, followed by a centrifugation. The cytoplasmic extract and the methods for obtaining same are known to those skilled in the art and are described in the prior art, for example in Suzuki, Keiko et al. “REAP: A two minute cell fractionation method.” BMC research notes vol. 3 294. 10 Nov. 2010, doi:10.1186/1756-0500-3-294 or in Gary Zieve and Sheldon Penman, Small RNA species of the HeLa cell: Metabolism and subcellular localization Cell, May 1976, Pages 19-31.

Typically, the cytoplasmic extract is obtained by incubation of the cell sample with a lysis buffer or a hypotonic buffer.

Incubation of the sample with the lysis buffer advantageously makes it possible to lyse or permeabilize the plasma membrane without permeabilizing the nuclear membrane.

Thus, for the purposes of the present invention, the term “lysis buffer” is understood to mean any buffer making it possible to lyse or permeabilize the plasma membrane without permeabilizing the nuclear membrane.

Those skilled in the art will be able to choose a suitable lysis buffer. Typically, a nonionic detergent may be used, such as described in the U.S. Pat. No. 5,637,465.

Those skilled in the art also know how to choose a suitable concentration of the buffer to make lysis or permeabilization of the plasma membrane possible without permeabilization of the nuclear membrane.

Typically, an anti-DNA antibody may be used by those skilled in the art for determining a suitable concentration of the buffer to make lysis or permeabilization of the plasma membrane possible without permeabilization of the nuclear membrane.

After treatment of the cells which have not initiated the death process with increasing concentrations of detergents, these cells are placed in the presence of an anti-DNA antibody. The nuclear exclusion of this antibody confirms the integrity of the nuclear membrane and thus makes it possible to determine the optimal concentration of detergent. Too high a concentration of detergent will permeabilize the nuclear membrane, allowing the antibody to penetrate into the nucleus and thus making it possible to detect nuclear DNA. This method is modeled on a method for detecting a protein which is located in the nucleus (Postfixation detergent treatment for immunofluorescence suppresses localization of some integral membrane proteins. Goldenthal K L, Hedman K, Chen J W, August J T, Willingham M C. J Histochem Cytochem. 1985 August; 33(8):813-20).

By way of illustration, the lysis buffer may be chosen from 4-(2-hydroxyethyl)-1-piperazineethanesulfonic acid, ethylenediaminetetraacetic acid, sodium chloride, saponins such as digitonin or saponin, Tween-20, NP40, Tergitol, Triton X-100, Igepal CA630, Empigen, or a combination.

According to one embodiment, the lysis buffer is a mixture of 4-(2-hydroxyethyl)-1-piperazineethanesulfonic acid, ethylenediaminetetraacetic acid, sodium chloride, and digitonin.

These buffers make it possible to lyse or permeabilize the plasma membrane without permeabilizing the nuclear membrane.

In one embodiment, the lysis buffer is a mixture of 50 mM 4-(2-hydroxyethyl)-1-piperazineethanesulfonic acid, 5 mM ethylenediaminetetraacetic acid, 150 mM sodium chloride, and 50 μg/ml digitonin.

According to one embodiment, the cytoplasmic extract can be obtained by the following steps:
incubation of the cell sample with a lysis buffer or a hypotonic buffer.
centrifugation or filtration of the lysed cell sample.

The step of centrifugation or of filtration makes it possible to separate the supernatant from the remainder of the cell debris.

The “fraction of the cytoplasmic extract” corresponds to an aliquot fraction of the cytoplasmic extract.

According to one embodiment, the fraction of the cytoplasmic extract can be obtained by the following steps:
incubation of the cell sample with a lysis buffer or a hypotonic buffer;
centrifugation or filtration of the lysed cell sample;
withdrawal of an aliquot fraction of the cytoplasmic extract in the supernatant obtained after the step of centrifugation or filtration.

According to one embodiment, the method for obtaining the cytoplasmic extract fraction may additionally comprise a step of dilution of said withdrawn aliquot fraction at least 5-fold in water, preferentially 10-fold in water.

Typically, the dilution step can be applied when the quantification method is a method for quantifying by PCR.

Advantageously, the inventors have demonstrated that it was possible to detect cell death on cytoplasmic extracts with the aid of primers targeting a sequence present in one copy on the nuclear genome.

Thus, and according to one embodiment, the method for quantifying cell death according to the present invention is characterized in that said at least one sequence is a DNA sequence present in one copy on said genomic DNA fragments.

The method for quantifying cell death according to the present invention also makes it possible to detect cell death on cytoplasmic extracts with the aid of primers targeting a sequence present in more than one copy in the nuclear genome.

By way of illustration, the method according to the present invention makes it possible to detect cell death on cytoplasmic extracts with the aid of primers targeting a sequence present in two copies in the nuclear genome.

Thus, according to one embodiment, the method for quantifying cell death according to the present invention is characterized in that said at least one sequence is a DNA sequence present in at least two copies on said genomic DNA fragments.

The sensitivity of the method according to the invention is increased proportionally to the number of repeat DNA sequences when these are used as markers for detecting and quantifying the quantity of cytoplasmic genomic DNA.

According to one embodiment, the method for quantifying cell death according to the present invention is characterized in that said at least one sequence is a repeat DNA sequence.

Genomic DNA typically includes repeat sequences which can be small interspersed nuclear elements (SINEs) or long interspersed nuclear elements (LINEs).

Among the SINEs, the Alu, MIR and MIR3 sequences can be chosen.

Among the LINEs, the LINE1 sequences can be chosen.

Thus, and according to one embodiment, the method for quantifying cell death according to the present invention is characterized in that said at least one repeat DNA sequence is chosen from SINEs such as Alu, MIR and MIR3, and LINEs such as LINE1.

The inventors have also demonstrated that it is possible to detect cell death on cytoplasmic extracts with the aid of several pairs of primers, each pair of primers targeting a DNA sequence present in the nuclear genome.

Thus, according to one embodiment, the method for quantifying cell death according to the present invention is characterized in that several different sequences are amplified simultaneously.

Those skilled in the art will be able to use any type of technique allowing detection and quantification of the genomic DNA sequences.

The detection/quantification of genomic DNA fragments can be carried out by quantitative PCR techniques or any other technique for detecting small amounts of DNA known to those skilled in the art.

According to one embodiment, the quantification and detection of fragmented genomic DNA is carried out by quantitative PCR (qPCR). The principle of qPCR is based on the possibility of determining the quantity of DNA template present in a sample in real time using an intercalating agent or a probe (Taqman®). The fluorescence emitted is directly proportional to the quantity of amplicons generated during the PCR reaction.

Those skilled in the art will be able to use any apparatus for implementing the qPCR technique. By way of illustration, the Light Cycler 480 real-time PCR reader system from the company Roche® can be used.

Thus, the method for quantifying cell death according to the present invention is characterized in that the detection/quantification is carried out by qPCR.

According to one embodiment, the quantification of the genomic DNA fragments is carried out by droplet digital PCR (ddPCR). ddPCR is a microfluidic PCR based on the partitioning of each sample into 20 000 1-nl droplets.

According to one embodiment, the quantification of the genomic DNA fragments is carried out by Nanostring technology. The principle of Nanostring is based on two key steps. Upstream, two probes are designed specifically for each target of interest. One of the probes, called capture probe, is coupled to a biotin which will be used to immobilize the molecules of interest on a support dedicated to the counting. The second probe, called “reporter”, is specific to the molecule of interest. It contains a series of 6 fluorochromes of 4 different colors, the arrangement of which defines a bar code which will be specific to each molecule of interest. It is this bar code which will enable the ultrasensitivity of this technique and hence the possibility of analyzing small amounts of biological material (LABEX DEEP Nanostring platform).

According to one embodiment, the quantification of the genomic DNA fragments is carried out by multiplex PCR or multiplexing. For implementation of a multiplex PCR, a set of several pairs of primers will be used so as to simultaneously amplify several sequences present in the genome.

According to one embodiment, a single sequence is detected and quantified using for example the sense primer 5′ CGCCTGGATCATGTCAAGTCA 3′ (SEQ ID NO: 1) and the antisense primer 5′ AGGCTAAGTTAGGGCCTCTGC 3′ (SEQ ID NO: 2) or the sense primer 5′ AACATAAGCTGAGGCCAGCCT 3′ (SEQ ID NO: 3) and the antisense primer 5′ GTGTCTACTGCCAACCTGTGC 3′ (SEQ ID NO: 4).

According to one embodiment, a sequence present in two copies is detected and quantified using the sense primer 5′ TCTCCACAACACTTAGTGGACAGT 3′ (SEQ ID NO: 5) and the antisense primer 5′ AGAGGAGGTGGTAGCTGGAGA 3′ (SEQ ID NO: 6).

According to one embodiment, multiplexing can be carried out using simultaneously, for example, the pair of primers SEQ ID NO: 1 and SEQ ID NO: 2 with the pair of primers SEQ ID NO: 3 and SEQ ID NO: 4.

According to one embodiment, the Alu sequence is detected and quantified using the sense primer 5′ AGGTGAAACCCCGTCTCTACT 3′ (SEQ ID NO: 7) and the antisense primer 5′ CCATTCTCCTGCCTCAGCCT 3′ (SEQ ID NO: 8).

According to one embodiment, the LINE1 sequence is detected and quantified using the sense primer 5′ GTCAGTGTGGCGATTCCTCAG 3′ (SEQ ID NO: 9) and the antisense primer 5′ AGTAATGGGATGGCTGGGTCAA 3′ (SEQ ID NO: 10) or using the sense primer 5′ AACAACAGGTGCTGGAGAGGA 3′ (SEQ ID NO: 11) and the antisense primer 5′ ATCGCCACACTGACTTCCACA 3′ (SEQ ID NO: 12).

According to one embodiment, the amount of amplified nucleic acid in said sample of nucleic acid will be compared with the amount of amplified nucleic acid of a control sample.

The term “control sample” is understood to mean a cell sample in which the process of cell death has not been initiated.

Typically, an increase in the amount of amplified nucleic acid in said “sample” compared to the amount of amplified nucleic acid of the “control sample” is indicative of cell death.

The quantification of cell death by the detection of DNA fragments in the cytoplasm of cells according to the present invention makes it possible to diagnose a pathology, to monitor the effects of a treatment on cell death, to obtain a prognosis of the pathology, to carry out a screening of compounds, and to optimize conditions of cell culture.

Thus, the present invention also relates to a method for monitoring the efficacy and/or the effect of a treatment on cell death, comprising the detection of cell death in a cell sample by the method according to the present invention.

The method applies to in vitro, in vivo and ex vivo conditions.

Typically, the present invention makes it possible to monitor the response of a patient to the treatment. The detection and the quantification of cell death are indicative of the efficacy, or lack thereof, of the treatment. The amount of amplified nucleic acid of the sample originating from the patient can be compared to the amount of amplified nucleic acid of a control sample, said control sample possibly being a sample from the patient obtained before administration of the treatment or a sample originating from a subject not suffering from the pathology. In general, an increase in the amount of amplified nucleic acid is synonymous with the effectiveness of the treatment, whereas the absence of significant variation may be synonymous with treatment failure.

The present invention also relates to a method for diagnosing a pathology involving a process of cell death, comprising the detection of cell death in a cell sample by the method according to the present invention.

Typically, the level of activation of cell death in a cell sample would be indicative of a pathology involving a process of cell death. The amount of amplified nucleic acid of the sample originating from the patient can be compared to the amount of amplified nucleic acid of a control sample, said control sample possibly being a sample originating from a subject not suffering from the pathology.

The present invention also relates to a method for the prognosis of a pathology involving a process of cell death, comprising the detection of cell death in a cell sample by the method according to the present invention.

The present invention also relates to a method for screening compounds, comprising:
the treatment of a cell sample with one or more compounds;
the detection of cell death in said sample by the method according to the present invention.

The method according to the invention will make it possible to determine the compound(s) triggering cell death.

A subject of the present invention is also a kit for the detection of cell death in a cell sample, comprising:
a lysis buffer or a hypotonic buffer capable of specifically lysing or permeabilizing the plasma membrane;
at least one pair of primers which amplify genomic DNA.

The lysis buffer advantageously makes it possible to lyse or permeabilize the plasma membrane without permeabilizing the nuclear membrane.

The lysis buffer and the primers are as described above.

The cell sample may be chosen from a sample of cells in in vitro culture, such as adherent cells or cells in suspension, a sample comprising circulating tumor cells, a sample comprising purified circulating tumor cells, a blood sample containing circulating cells or any sample comprising cells that have initiated a process of cell death, such as a plasma sample, a urine sample or a saliva sample.

Likewise advantageously, the primers will be specific to the species studied.

### Examples

In the examples which follow the materials and methods detailed below were used:

Method

A protocol was developed for measuring the cell death in a given sample.

Starting from cells untreated or treated with different drugs which trigger the death thereof, the cells are lysed using a buffer in order to release into the medium the small fragments of DNA resulting from its degradation.

A step of centrifugation or of filtration makes it possible to separate the supernatant containing the fragments of DNA resulting from its degradation from the rest of the cell debris. An aliquot fraction of the supernatant is withdrawn, i.e. the fraction of cytoplasmic extract which will possibly be diluted depending on the method for quantifying cell death used. Then, a PCR is carried out on the samples using primers which specifically amplify repeat sequences dispersed throughout the genome, or using primers which target a sequence in one copy or two copies on the genome.

Alternatively, the method for quantifying cell death may be carried out on a cytoplasmic extract obtained from cells lysed directly in the culture medium.

On completion of the method for quantifying cell death, the results are analyzed relative to a predetermined control condition. The final result may be obtained in two to three hours.

Primers

Primers enabling the detection and the quantification of a single sequence:

(SEQ ID NO: 1)
5′ CGCCTGGATCATGTCAAGTCA 3′
and
(SEQ ID NO: 2)
5′ AGGCTAAGTTAGGGCCTCTGC 3′
or
(SEQ ID NO: 3)
5′ AACATAAGCTGAGGCCAGCCT 3′
and
(SEQ ID NO: 4)
5′ GTGTCTACTGCCAACCTGTGC 3′.

Primers enabling the detection and the quantification of a sequence present in two copies per genome:

(SEQ ID NO: 5)
5′ TCTCCACAACACTTAGTGGACAGT 3′
and
(SEQ ID NO: 6)
5′ AGAGGAGGTGGTAGCTGGAGA 3′.

Primers enabling the detection and the quantification of the Alu repeat sequence:

(SEQ ID NO: 7)
5′ AGGTGAAACCCCGTCTCTACT 3′
(SEQ ID NO: 8)
5′ CCATTCTCCTGCCTCAGCCT 3′.

Primers enabling the detection and the quantification of the LINE1 repeat sequence:

(SEQ ID NO: 9)
5′ GTCAGTGTGGCGATTCCTCAG 3′
and
(SEQ ID NO: 10)
5′ AGTAATGGGATGGCTGGGTCAA 3′
or
(SEQ ID NO: 11)
5′ AACAACAGGTGCTGGAGAGGA 3′
and
(SEQ ID No: 12)
5′ ATCGCCACACTGACTTCCACA 3′.

Cell Culture

OCI-AML3 cells (in suspension) are obtained from acute myeloid leukemia. They are cultured in RPMI-1640 medium (Sigma-Aldrich) supplemented with penicillin-streptomycin (Sigma-Aldrich) and 10% fetal calf serum (FCS—Gibco) at 37° C. with 5% CO₂.

HepG2 (adherent) cells are obtained from human liver carcinoma. They are cultured in Dulbecco's Modified Eagle Medium-High Glucose medium (DMEM—Sigma-Aldrich) supplemented with penicillin-streptomycin (Sigma-Aldrich) and 10% fetal calf serum (FCS—Gibco) at 37° C. with 5% CO₂.

MDA-MB-231 (adherent) cells are epithelial mammary tumor cells. They are cultured in Dulbecco's Modified Eagle Medium-High Glucose medium (DMEM—Sigma-Aldrich) supplemented with penicillin-streptomycin (Sigma-Aldrich) and 10% fetal calf serum (FCS—Gibco) at 37° C. with 5% CO2.

MOLM14 cells (in suspension) are obtained from acute myeloid leukemia. They are cultured in RPMI-1640 medium (Sigma-Aldrich) supplemented with penicillin-streptomycin (Sigma-Aldrich) and 10% fetal calf serum (FCS—Gibco) at 37° C. with 5% CO2.

The HeLa cells were cultured in Dulbecco's Modified Eagle Medium-Low Glucose medium (DMEM—Sigma-Aldrich) supplemented with penicillin-streptomycin (Sigma-Aldrich) and 10% fetal calf serum (FCS—Gibco) at 37° C. with 5% CO2.

Inoculation and Treatment

Cells in suspension (for example: OCI-AML3) are inoculated in a 6-well plate at 200 000 cells/ml. The cells are then left untreated or treated with different drugs at different concentrations and incubated for 16 hours.

Adherent cells are inoculated at between 20 000 and 30 000 cells/well (for example: MDA-MB-231, HepG2) in a 48-well plate in 250 μl of medium. The cells are then left untreated or treated with different drugs at different concentrations and incubated for 24 hours.

qPCR

For Implementation of the qPCR, the Cytoplasmic Fraction Obtained Will be Diluted 10-Fold in Water.

The kit used is SYBR qPCR Premix Ex Taq II (Takara). The samples were diluted beforehand to 1/10^{th} in water. Once the master mix has been prepared (as described in the table below), 6 μl are distributed in each well of the PCR plate. Then 4 μl of sample are added. The PCR is carried out in a Light Cycler 480 real-time PCR reader system (Roche). The amplification program is as follows: 95° C. for 30 seconds, followed by 40 cycles consisting of two steps of 95° C. for 5 seconds and 60° C. for 20 seconds.

TABLE 1
Preparation of the master mix (MM)

| | Concentration | Concentration | Volume for 1 |
|---|---|---|---|
| Reagents | Stock solution | Final | Reaction (μl) |
| Ex taq II 2× mix | 2 × | 1 × | 5.00 |
| Primer F-R | 10 | 300 | 0.30 |
| ROX | 50X | | 0.12 |
| H₂O | | | 0.58 |
| Total volume (μl) | | | 6.00 |

Caspase Glo

Untreated cells (NT) and cells having undergone a treatment (TTT) for inducing cell death were used for these assays. Under each of the NT and TTT conditions, 100 000-10 000-1000-100 or 10 cells are transferred to a 96-well plate in 50 μl of medium (in triplicates). Next, 50 μl of Caspase Glo 3/7 reagent are added. After incubation for 1 hour, the light emission resulting from the cleavage of the substrate by caspases 3 and 7 is determined using a luminometer.

Annexin V/Propidium Iodide (PI) Labeling—Biolegend

Untreated cells (NT) and cells having undergone a treatment (TTT) for inducing cell death were used for these assays.

The cells are washed with PBS then resuspended in 1× binding buffer at a concentration of 1×10⁶ cells/ml. 10 μl of Annexin V Pacific Blue and 10 μl of propidium iodide (Biolegend kit) are added to 200 μl of this cell suspension, which is then incubated for 15 minutes in darkness and at ambient temperature. After centrifugation at 300 g for 5 min, the supernatant is gently aspirated and 500 μl of 1× binding buffer are added.

These cells are then used in flow cytometry for determining the effect of the treatment and the number of cells in apoptosis. These cells are sorted depending on their annexin V and propidium iodide statuses in order to then carry out a PCR.

ddPCR (for Droplet Digital PCR)

Each ddPCR reaction is conducted optimally in about 20 000 droplets of 1 nl volume.

Samples and Preparation of the ddPCR Mix.

Cytoplasmic extracts obtained from untreated cells and cells treated to induce cell death were used as samples.

A ddPCR reaction mix (24 μl) requires 11 μl of 2× “EvaGreen ddPCR Supermix” (Bio-Rad), 0.22 μl of primers (sense and antisense, each at 200 nM final), 4 μl of sample and 6.78 μl of water.

Generation of Droplets

The droplets are generated by the QX200 DropletGenerator (Bio-Rad) by emulsifying 20 μl of ddPCR mix and 20 μl of oil in the wells of DG8 cartridges (Bio-Rad). The droplet/oil mixture is then transferred into a 96-well plate which is sealed using a “PX1 PCR Plate Sealer” (Bio-Rad).

Amplification

Amplification is carried out in a T100 thermal cycler (Bio-Rad) following the program: Enzyme activation: 95° C. for 5 min; 40 cycles: denaturation at 95° C. for 30 s then extension at 60° C. for 1 min. Signal stabilization: 4° C. for 5 min then 90° C. for 5 min.

Droplet Reading

The plate is then read by the QX200 Droplet Reader (Bio-Rad). The results are then exported and the data analysed with QuantaSoft software (Bio-Rad).

### Example 1: Detection of Cell Death on Adherent Cells in Culture (HepG2 and MDA-MB-231) or Cells in Suspension (OCI-AML3)

HepG2 Adherent Cells in Culture

FIG. 1 represents the level of activation of cell death realized on cytoplasmic extracts of HepG2 cells obtained using various lysis buffers in the method according to the present invention.

30 000 cells are inoculated and then are left untreated or treated for 18 h with 1 μM doxorubicin. The cells are then lysed in a buffer containing different detergents: 0.075% Tween-20, 0.0075% Triton X-100, 0.037% Empigen and 0.33% Tergitol. The cytoplasmic extracts are centrifuged and then a fraction of the supernatant is withdrawn and diluted (10×) in water. qPCR is then carried out on said fraction.

The pair of primers of SEQ ID NO: 9 and SEQ ID NO: 10 was used on the fractions of cytoplasmic extracts obtained after lysis of the cells with 0.33% Tergitol. The pair of primers of SEQ ID NO: 7 and SEQ ID NO: 8 was used on the fractions of cytoplasmic extracts obtained after lysis of the cells with 0.075% Tween-20, 0.0075% Triton X-100 and 0.037% Empigen.

OCI-AML3 Cells in Suspension

FIG. 2 represents the level of activation of cell death realized on cytoplasmic extracts of OCI-AML3 cells obtained using various detergents in the method according to the present invention.

The cells are left untreated or treated for 18 h with 1 μM doxorubicin. After centrifugation of 5000 cells, the latter are lysed in a buffer containing different detergents: 0.1% Tergitol, 0.1% Empigen, 150 μg/ml digitonin, 0.1% NP40. qPCR is then carried out on fractions of cytoplasmic extracts using the pair of primers of SEQ ID NO: 9 and SEQ ID NO: 10.

FIG. 3 represents the level of activation of cell death realized on cytoplasmic extracts of OCI-AML3 cells obtained using a lysis buffer produced with different detergents in the method according to the present invention.

5000 cells are left untreated or treated for 18 h with 1 μM doxorubicin and then lysed directly in a buffer containing different detergents: 0.1% Tergitol, 0.5% NP40, 0.01% Triton X-100, 50 μg/ml digitonin. qPCR is then carried out on fractions of cytoplasmic extracts using the pair of primers of SEQ ID NO: 7 and SEQ ID NO: 8. The results of the experiment presented are means of three independent experiments.

MDA-MB-231 Adherent Cells in Culture

FIG. 4 represents the level of activation of cell death realized on cytoplasmic extracts of MDA-MB-231 cells obtained using a lysis buffer produced with different detergents in the method according to the present invention.

20 000 cells are left untreated or treated for 18 h with 1 μM doxorubicin and then lysed directly in a buffer containing different detergents: 0.1% Tergitol, 0.5% Igepal CA630, 0.01% Triton X-100 and 0.5% Tween-20. qPCR is then carried out on fractions of cytoplasmic extracts using the pair of primers of SEQ ID NO: 7 and SEQ ID NO: 8.

The method according to the present invention advantageously makes it possible to detect cell death and hence to measure the level of activation thereof in samples of adherent cells and of cells in suspension.

### Example 2: Determination of the Sensitivity of the Method for Detecting Cell Death by PCR According to the Present Invention Compared to the Caspase Glo 3/7 Method of the Prior Art

MOLM14 cells are treated for 16 h with 1 μM etoposide. Cell death is determined on a sample of cells ranging from 10 to 10 000 cells using the Caspase Glo technique. In parallel, a qPCR is carried out on a cytoplasmic extract originating from an identical number of cells (from 10 to 10 000 cells) with the pair of primers of SEQ ID NO: 7 and SEQ ID NO: 8. The results of the experiment presented in FIG. 5 are means of three independent experiments.

Comparison of the method according to the present invention with a method of the prior art (Caspase Glo) makes it possible to advantageously demonstrate the improved sensitivity and the lower detection threshold of the method according to the present invention.

### Example 3: Determination of the Sensitivity of the Method for Detecting Cell Death According to the Present Invention Compared to Flow Cytometry (Annexin V, Propidium Iodide Labeling) of the Prior Art

OCI-AML3 Cells Untreated or Treated with Increasing Concentrations of Etoposide

qPCR on a Cytoplasmic Extract Obtained from Cells Lysed after Centrifugation.

OCI-AML3 cells are left untreated (NT) or treated for 16 h with increasing concentrations of etoposide (0-7.5-15 or 30 μM).

The cells are labeled using the Pacific Blue™ Annexin V kit (Biolegend) according to the supplier's recommendations and then analyzed by flow cytometry (MACSQuant VYB—Miltenyi Biotec). The percentage of positive apoptotic cells (annexin V) is determined.

In parallel, a qPCR is carried out on a cytoplasmic extract obtained from 10 000 cells lysed after centrifugation with the pair of primers of SEQ ID NO: 9 and SEQ ID NO: 10. The results are presented in FIG. 6.

qPCR Carried Out on a Cytoplasmic Extract Obtained from Cells Lysed Directly in the Culture Medium.

OCI-AML3 cells are left untreated (NT) or treated for 16 h with increasing concentrations of etoposide (0-7.5-15 or 30 μM).

The cells are labeled using the Pacific Blue™ Annexin V kit (Biolegend) according to the supplier's recommendations and then analyzed by flow cytometry (MACSQuant VYB—Miltenyi Biotec). The percentage of positive apoptotic cells (annexin V) is determined.

In parallel, a PCR is carried out on a cytoplasmic extract obtained from 10 000 cells lysed directly in the culture medium after centrifugation, with the pair of primers of SEQ ID NO: 9 and SEQ ID NO: 10. The results are presented in FIG. 7.

OCI-AML3 Cells Left Untreated or Treated with Increasing Concentrations of Bortezomib

qPCR on a Cytoplasmic Extract Obtained from Cells Lysed after Centrifugation.

OCI-AML3 cells are left untreated (NT) or treated for 16 h with increasing concentrations of bortezomib (0-0.125 or 0.25 μM).

The cells are labeled using the Pacific Blue™ Annexin V kit (Biolegend) according to the supplier's recommendations and then analyzed by flow cytometry (MACSQuant VYB—Miltenyi Biotec). The percentage of positive apoptotic cells (annexin V) is determined.

In parallel, a PCR is carried out on a cytoplasmic extract obtained from 10 000 cells lysed after centrifugation with the pair of primers of SEQ ID NO: 9 and SEQ ID NO: 10. The results are presented in FIG. 8.

qPCR Carried Out on a Cytoplasmic Extract Obtained from Cells Lysed Directly in the Culture Medium

OCI-AML3 cells are left untreated (NT) or treated for 16 h with increasing concentrations of bortezomib (0-0.125 or 0.25 μM).

The cells are labeled using the Pacific Blue™ Annexin V kit (Biolegend) according to the supplier's recommendations and then analyzed by flow cytometry (MACSQuant VYB—Miltenyi Biotec). The percentage of positive apoptotic cells (annexin V) is determined.

In parallel, a PCR is carried out on a cytoplasmic extract obtained from 10 000 cells lysed directly in the culture medium after centrifugation, with the pair of primers of SEQ ID NO: 9 and SEQ ID NO: 10. The results are presented in FIG. 9.

MOLM14 Cells Left Untreated or Treated with Increasing Concentrations of Etoposide

qPCR on a Cytoplasmic Extract Obtained from Cells Lysed after Centrifugation

MOLM14 cells are left untreated (NT) or treated for 16 h with increasing concentrations of etoposide (0-0.62-1.25-2.5-5 or 10 μM).

The cells are labeled using the Pacific Blue™ Annexin V kit (Biolegend) according to the supplier's recommendations and then analyzed by flow cytometry (MACSQuant VYB—Miltenyi Biotec). The percentage of positive apoptotic cells (annexin V) is determined.

In parallel, a PCR is carried out on a cytoplasmic extract obtained from 10 000 cells lysed after centrifugation with the pair of primers of SEQ ID NO: 7 and SEQ ID NO: 8.

The sensitivity of the method using PCR according to the present invention is far greater than that of flow cytometry. In addition, the method using flow cytometry reaches a detection plateau starting from a concentration of 2.5 μM of etoposide, which is not the case for the PCR method. The results are presented in FIG. 10.

Comparison of the method according to the present invention with a method of the prior art (annexin V, propidium iodide labeling) makes it possible to advantageously demonstrate the improved sensitivity and the lower saturability of the method according to the present invention.

The method also makes it possible to detect cell death directly after lysis in the culture medium, this being done with far greater sensitivity than with the techniques of the prior art.

OCI-AML3 Cells Treated with 1 μM Doxorubicin

OCI-AML3 cells are left untreated or treated for 16 h with 1 μM doxorubicin.

The cells are labeled using the Pacific Blue™ Annexin V kit (Biolegend). 1000 treated or untreated double negative cells (dn population—annexin V negative-propidium iodide negative) are sorted. These results are shown in FIG. 11A.

A PCR is carried out on a cytoplasmic extract originating from these 1000 treated (TTT) or untreated (NT) (AV−/PI−) cells, using the pair of primers of SEQ ID NO: 9 and SEQ ID NO: 10. The results of the experiment presented are means of three independent experiments. The results are presented in FIG. 11B.

It appears that the method according to the present invention makes it possible to detect the appearance of cell death on cells considered to be double negative for annexin V/propidium iodide labeling by the prior art test in view of the sensitivity threshold which favors the present invention. The present invention enables earlier detection of cell death than with the standard test of the prior art.

Isolated Cells

MOLM14 cells are left untreated or treated for 16 h with 2.5 μM etoposide.

The cells are labeled using the Pacific Blue™ Annexin V kit (Biolegend). For each sub-population, 1 cell is sorted and a ddPCR (droplet digital) is carried out on a cytoplasmic extract using the pair of primers of SEQ ID NO: 9 and SEQ ID NO: 10. The level of activation of cell death is determined and compared with the signals obtained from the cytoplasmic extract of an untreated cell negative for propidium iodide/annexin V labeling.

The results as presented in FIG. 12 represent the mean of the results obtained with 5-6 cells.

The method according to the invention makes it possible to detect cell death on a cell sample and thus offers optimized sensitivity compared to methods of the prior art.

### Example 4: Detection of Cell Death Realized on Cytoplasmic Extracts from Different Cell Lines Using Primers Targeting a Sequence Present in One Copy or in Two Copies on the Genome

FIG. 13 represents the level of activation of cell death realized on cytoplasmic extracts from the cell lines indicated on the abscissa axis (MDA-MB-231, Molm14, HeLa, OCI-AML3).

The MDA-MB-231 cells are left untreated (NT) or treated (TTT) for 24 h with 200 nM staurosporine. The Molm14 cells are left untreated (NT) or treated (TTT) for 16 h with 0.5 μM doxorubicin. The HeLa cells are left untreated (NT) or treated (TTT) for 24 h with 400 nM staurosporine. The OCI-AML3 cells are left untreated (NT) or treated (TTT) for 16 h with 1 μM doxorubicin. After action of the lysis buffer, qPCR is carried out on the cytoplasmic extracts, diluted 10-fold, using the pair of primers of SEQ ID NO: 1 and SEQ ID NO: 2.

FIG. 14 represents the level of activation of cell death realized on cytoplasmic extracts from the cell lines indicated on the abscissa axis (MDA-MB-231, Molm14, HeLa, OCI-AML3).

The MDA-MB-231 cells are left untreated (NT) or treated (TTT) for 24 h with 200 nM staurosporine. The Molm14 cells are left untreated (NT) or treated (TTT) for 16 h with 0.5 μM doxorubicin. The HeLa cells are left untreated (NT) or treated (TTT) for 24 h with 400 nM staurosporine. The OCI-AML3 cells are left untreated (NT) or treated (TTT) for 16 h with 1 μM doxorubicin. After action of the lysis buffer and centrifugation, qPCR is carried out on the cytoplasmic extracts, diluted 10-fold, using the pair of primers of SEQ ID NO: 5 and SEQ ID NO: 6.

Advantageously, the method according to the invention makes it possible to detect cell death by detection and amplification of a DNA sequence present in one copy on the genome.

### Example 5: Detection of Cell Death Realized on Cytoplasmic Extracts by Multiplex

FIG. 15 represents the level of activation of cell death realized on cytoplasmic extracts of the OCI-AML3 cell line.

The cells are left untreated (NT) or treated (TTT) for 24 h with 10 μM aracytine [cytarabine]. After centrifugation, the cells are lysed. qPCR is then carried out on the cytoplasmic extracts, diluted 10-fold, using simultaneously the pair of primers of SEQ ID NO: 1 and SEQ ID NO: 2 with the pair of primers of SEQ ID NO: 3 and SEQ ID NO: 4.

The method for detecting cell death according to the present invention can advantageously be implemented by a multiplexing technique.

Thus, and advantageously, the inventors have developed a method for detecting cell death which is sensitive, simple and rapid, has a moderate cost and works on adherent cells or cells in suspension. This method has increased sensitivity, a much lower detection threshold, and a lower saturability compared to the methods of the prior art.

### Example 6: Detection of the Presence of DNA Fragments in the Cytoplasmic Fraction which are Obtained after Extraction with a Lysis Buffer Described in the Method According to the Invention and are Detected by Capillary Electrophoresis in Cells Treated by a Drug Inducing the Death Thereof (Staurosporine) or Left Untreated (NT)

MDA-MB-231 cells were inoculated in 6 T175s at an amount of 3 million cells per 35 ml of DMEM high glucose medium (10% FCS, P/S). The next day, the MDA-MB-231 cells were treated with 50 nM and 100 nM staurosporine. The medium of the 2 untreated T175s was changed. After 24 h of treatment, the MDA-MB-231 cells were lysed with 2 ml of lysis buffer added to the T175s for 15 min at ambient temperature, then 2 ml were transferred into a tube and centrifuged at 2000 g for 5 min. The supernatant was then treated with RNaseA (20 μg/ml) and then with Proteinase K (used at 100 ug/ml), and placed at 70° C. for 15 min. The lysis buffer is a mixture of 50 mM 4-(2-hydroxyethyl)-1-piperazineethanesulfonic acid, 5 mM ethylenediaminetetraacetic acid, 150 mM sodium chloride, and 50 μg/ml digitonin.

1/10 volume of sodium acetate (NaAc) (3 M, pH 5.2) was then added. DNA was precipitated with 1 volume of isopropanol. The samples were left at ambient temperature for 15 min and then centrifuged at 20 000 g for 20 min at +4° C. The supernatant was aspirated and the pellet was washed with 75% ethanol. The pellet was dried and then resuspended in 30 μl of TE at pH 8 and analyzed in a fragment analyzer.

The presence of DNA in the cytoplasmic fraction of MDA-MB 231 cells untreated or treated for 24 h with 50 or 100 nM staurosporine was analyzed and was as shown in FIG. 16.

DNA fragments, a marker of cell death, are detected in the cytoplasmic fraction of the cell samples treated with a drug inducing the death of said cells (staurosporine). There is very little, if any, fragmented DNA in the cytoplasmic fraction of the cell sample which has not been subjected to treatment inducing cell death.

1. A method for quantifying cells which have initiated a cell death process in a cell sample, comprising
obtaining a cytoplasmic extract from the cell sample; and
quantifying genomic DNA in the cytoplasmic extract by amplifying at least one sequence present in genomic DNA fragments of nuclear origin in the cytoplasmic extract.

2. (canceled)

3. The method as claimed in claim 1, wherein the cytoplasmic extract is obtained by incubation of the cell sample with a lysis buffer or a hypotonic buffer.

4. The method as claimed in claim 1, wherein the at least one sequence is a DNA sequence present in one copy per genome.

5. The method as claimed in claim 1, wherein the at least one sequence is a DNA sequence present at least twice per genome.

6. The method as claimed in claim 1, wherein the at least one sequence is a repeat DNA sequence.

7. The method as claimed in claim 6, wherein the repeat DNA sequence is chosen from SINEs and LINEs.

8. The method as claimed in claim 1, wherein the step of amplifying is carried out by a PCR technique or Nanostring technology.

9. The method as claimed in claim 8, wherein the PCR technique is quantitative PCR, droplet digital PCR or multiplex PCR.

10. A method for monitoring the efficacy of a treatment involving cell death in a subject, comprising detecting cell death in a cell sample from the subject by the method of claim 1.

11. A method for diagnosing a pathology involving a process of cell death in a subject, comprising detecting cell death in a cell sample from the subject by the method of claim 1.

12. A method for screening compounds, comprising:
treating a cell sample with at least one compound; then
detecting cell death in said cell sample by the method of claim 1.

13. A kit for the detection of cell death in a cell sample, comprising:
a lysis buffer or a hypotonic buffer capable of specifically lysing or permeabilizing the plasma membrane; and
at least one pair of primers which amplify genomic DNA.


SEQUENCE LISTING (12 sequences)

SEQ ID NO: 1 - DNA, 21 bp, Artificial Sequence
  Synthetic Unique Sequence forward primer
cgcctggatcatgtcaagtca

SEQ ID NO: 2 - DNA, 21 bp, Artificial Sequence
  Synthetic Unique sequence reverse primer
aggctaagttagggcctctgc

SEQ ID NO: 3 - DNA, 21 bp, Artificial Sequence
  Synthetic Unique sequence forward primer
aacataagctgaggccagcct

SEQ ID NO: 4 - DNA, 21 bp, Artificial Sequence
  Synthetic Unique sequence reverse primer
gtgtctactgccaacctgtgc

SEQ ID NO: 5 - DNA, 24 bp, Artificial Sequence
  Synthetic Two copies forward primer
tctccacaacacttagtggacagt

SEQ ID NO: 6 - DNA, 21 bp, Artificial Sequence
  Synthetic Two copies reverse primer
agaggaggtggtagctggaga

SEQ ID NO: 7 - DNA, 21 bp, Artificial Sequence
  Synthetic ALU forward primer
aggtgaaaccccgtctctact

SEQ ID NO: 8 - DNA, 20 bp, Artificial Sequence
  Synthetic ALU reverse primer
ccattctcctgcctcagcct

SEQ ID NO: 9 - DNA, 21 bp, Artificial Sequence
  Synthetic LINE1 forward primer
gtcagtgtggcgattcctcag

SEQ ID NO: 10 - DNA, 22 bp, Artificial Sequence
  Synthetic LINE1 reverse primer
agtaatgggatggctgggtcaa

SEQ ID NO: 11 - DNA, 21 bp, Artificial Sequence
  Synthetic LINE1 forward primer
aacaacaggtgctggagagga

SEQ ID NO: 12 - DNA, 21 bp, Artificial Sequence
  Synthetic LINE1 reverse primer
atcgccacactgacttccaca